(19) United States
(12) Patent Application Publication
(10) Pub. No.: US 2006/0018842 A1
(43) Pub. Date: Jan. 26, 2006

(54) Composition and method for delivery of phytochemicals

(76) Inventor: Gary Blumenthal, Wynnewood, PA (US)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 11/166,543

(22) Filed: Jun. 24, 2005

Related U.S. Application Data
(60) Provisional application No. 60/582,948, filed on Jun. 25, 2004.

(51) Int. Cl. A61K 36/82; A61K 9/68

(52) U.S. Cl. 424/48; 424/729

(57) ABSTRACT

Anti-microbial and anti-cancer phytochemicals can be delivered with a composition comprising an phytochemical, in which the composition allows the controlled release of the phytochemical upon administration of the composition to a subject. The phytochemicals can be delivered as a nutritional supplement, or for the treatment or prevention of cancer or microbial infections.

### CROSS-REFERENCE TO RELATED APPLICATIONS

This application claims the benefit of U.S. provisional patent application Ser. No. 60/582,948, filed on Jun. 25, 2004, the entire disclosure of which is herein incorporated by reference

### FIELD OF THE INVENTION

This invention relates to compositions comprising one or more phytochemicals for use as a nutritional supplement or for administration to a subject suffering from cancer or microbial infection.

### BACKGROUND

Consumption of certain naturally-occurring substances may confer disease resistance or provide a therapeutic effect. Individuals consuming a diet high in fruits and vegetables are believed to have a lower risk of developing cancer, and also appear to be better able to overcome microbial infections. For example, it has been reported that a diet high in vegetables and fruits could prevent at least 20% of all cancers, and a reduced risk of developing cancers of the mouth, pharynx, esophagus, stomach, colon, and rectum.

Plant-derived agents or “phytochemicals” which have anti-microbial or anti-cancer effect may target molecules inside the mammalian cell that regulate the cell cycle, cellular senescence, and apoptosis. Different categories of phytochemicals with potential anti-microbial or anti-cancer effect can be derived from various plant sources. For example, green tea catechins, also known as polyphenols, are widely known as an anticancer agent. Further, tea catechins have been shown to have antibacterial activity against a variety of food-borne pathogenic bacteria.

In vitro studies showed that green tea causes reversible G1 arrest of the cell cycle by inhibition of retinoblastoma protein (Rb) phosphorylation in oral leukoplakia. The most potent component of green tea polyphenol, epigallocatechin-3-gallate (EGCG), has been shown to induce growth inhibition of transformed cells by activating apoptosis, while normal cell growth was not affected. EGCG alone or as part of a mixture of green tea polyphenols is able to induce apoptosis in oral squamous carcinoma cells, while at the same time normal human epidermal keratinocytes survive. Green tea polyphenols have also been found to induce apoptosis in many types of tumor cells, including oral cancer cells. Based on such in vitro experiments, various mechanisms have been proposed to account for the cancer chemo-preventive activity of the green tea polyphenols. However, the relevance of these mechanisms in vivo remains in question, due to an incomplete understanding of the bioavailability of the polyphenolic compounds in traditional tea-drinks or nutritional supplements.

Another phenolic compound, [6]-paradol, is derived from ginger root and certain Zingiberaceae plants. [6]-paradol protected mouse skin from a tumor inducing agent, and showed dose-dependent cytotoxicity in an oral carcinoma cell line (KB), with specific features of caspase-3-mediated apoptosis. Viable KB cells were reduced in number to less than 50% of untreated control when incubated with [6]-paradol for 48 h. In addition, an ethanolic extract of ginger decreased the number of tumors in a Sencar mouse skin tumor model. Curcumin, a yellow coloring agent in turmeric (Zingiberaceae family), has also has been shown to exert anti-carcinogenic effects. More recently, curcumin has been shown to reduce the growth and metastasis of human breast cancer xenografts in mice.

Cinnamic acid, found in coffee, yields chlorogenic acid (CGA). CGA is a phenolic compound which reduces serum cholesterol and triglycerides. CGA also induced caspase-3-dependent apoptosis in oral cancer, while normal cells were unaffected.

Garlic has long been used to treat various illnesses, and its anticancer potential has recently been investigated. In a 7,12-dimethylbenz[a]anthracene (DMBA)-induced hamster buccal pouch carcinogenesis model, water extracts of fresh garlic induced apoptosis of malignant cells and completely prevented the onset of oral carcinoma.

Narcotics isolated from plants also induce apoptosis in oral squamous cell carcinoma (OSCC) cells. For example, codeinone (a derivative of the opioid analgesic codeine) was shown to target the mitochondria and cause caspase-3-dependent apoptosis. The cytotoxicity caused by codeinone was selectively higher in oral tumor cells.

Carotenoids are another important class of therapeutic phytochemical. There are more than 600 known carotenoids, of which approximately 25 are also present in human serum (nine are metabolites) and 14 are present in human tissues. The most common carotenoids found in human serum are lycopene, lutein, β-carotene, α-carotene, and β-crytoxanthin.

The primary sources of lycopene in the diet include tomatoes, apricots, papaya, and other yellow fruits. Consumption of tomato-containing foods is inversely correlated with the incidence of some systemic neoplasms. In particular, lycopene and other carotenoid-rich foods also are inversely related to upper gastrointestinal tract neoplasms, including oral cancer. Laboratory studies also showed that lycopene blocked IGF-1 stimulated proliferation in the breast cancer cell line MCF7 by interfering with IGF-1 signaling. Lycopene specifically induced a key protein for gap junction formation, called “connexin 43,” and inhibited the proliferation of the oral cancer cell line KB-1 in G1 phase. At physiological lycopene concentrations, KB-1 cells were inhibited to approximately 10% of control cell numbers.

Retinoids are the natural and synthetic derivatives of vitamin A. The retinoids in the body originate from retinyl esters, carotenoids, and retinal in diets. The role of retinoids in preventing and treating oral cancer has recently been reviewed, and is known to those of skill in the art.

However, in developed countries, the typical human diet is no longer rich in sources of phytochemicals. Moreover, normal dietary sources of phytochemicals are likely not ideally suited for delivery of these substances to a subject in order to obtain maximum bioavailability. Moreover, normal dietary sources of phytochemicals do not contain ideal combinations of phytochemicals to produce the maximum therapeutic or nutritional benefit. What is needed, therefore, is a composition for delivering phytochemicals in a controlled manner and/or in a desirable combinations as a dietary supplement, or as a therapeutic for the treatment or prevention of disease.

### SUMMARY OF THE INVENTION

Efficient delivery of anti-cancer and anti-microbial phytochemicals to subjects can be accomplished with a controlled release composition, or a composition comprising combinations of phytochemicals.

The invention thus provides a composition comprising a phytochemical and an pharmaceutically acceptable controlled release carrier. The invention also provides a composition comprising particular combinations of phytochemicals.

The invention also provides a method of providing a subject with a phytochemical as a nutritional supplement, comprising the step of administering a composition of the invention to the subject.

The invention further provides a method of treating cancer or microbial infection in a subject in need of such treatment, or of inhibiting the growth of cancer cells in a subject, comprising the step of administering a composition of the invention to the subject, wherein the composition delivers an effective amount of the phytochemical.

### BRIEF DESCRIPTION OF THE FIGURES

FIG. 1 shows the structural formulae of common antimicrobial and anticancerous phytochemicals.

FIG. 2 shows the structural formulae of the principal components of green tea catechins, which are also known as green tea polyphenols.

FIG. 3 shows the structural formula of the principal components of black tea polyphenols, which are also known as the theaflavins.

### DETAILED DESCRIPTION

Efficient delivery of anti-microbial and anti-cancer phytochemicals can be accomplished with a composition comprising at least one phytochemical, in which the composition allows the controlled release of the phytochemical upon administration of the composition to a subject. A composition of the invention thus comprises one or more phytochemicals and a pharmaceutically acceptable controlled release carrier. The composition of the invention can also comprise phytochemicals in a particular combination. The phytochemicals in the present compositions can be isolated phytochemicals.

As used herein, an “isolated” substance is a substance which is synthetic, or which is altered or removed from the natural state through human intervention. For example, a phytochemical which is partially or completely separated from the coexisting materials of its natural state is considered to be “isolated” for purposes of this invention. Powdered or dried plant material or plant extracts comprising phytochemicals are considered to be “isolated” phytochemicals. An isolated substance can exist in substantially purified form, or can exist in a non-native environment such as, for example, a cell or organism into which the substance has been introduced.

As used herein, a “subject” is any mammal, for example a murine, lapine, porcine, ovine, bovine, equine, feline, canine or primate mammal. In particular, the subject can be a primate mammal, for example a human. A subject can be, but is not necessarily, suffering from cancer or a microbial infection.

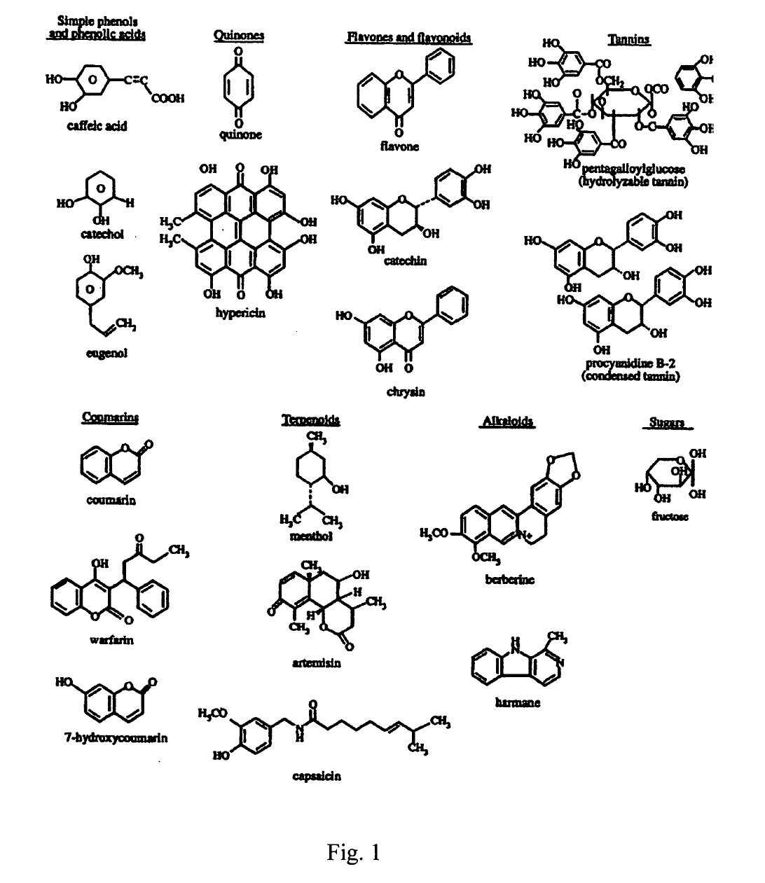

Phytochemicals for use in the present compositions include carotenoids (e.g., α-carotene, β-carotene, lutein, lycopene) found in tomatoes and other yellow/orange vegetables; isothiocyanates (e.g., sulphoraphane) found in cruciferous vegetables such as cabbage and broccoli; glucosinolates (e.g., glucobracinin and sinigrin) found in cruciferous vegetables such as brussel sprouts; sulfides (e.g., allyl sulfide), found in garlic, onions, scallions and broccoli; diarylhepanoids (e.g., curcumin) found in ginger and turmeric; saponins found in soybeans and other legumes; capsaicin found in red pepper and chili pepper; phenols/phenolics such as tea catechins, cinnamic acid from coffee, ellagic acid from berries, walnut and pecans, and resveratal (a phytoestrogen found in wine and grapes); and the flavonoids such as flavones (e.g., luteolin) found in apple skin and celery, acanthocyanins (e.g., cyanidin) found in berries, flavanons (e.g., hesperitin) found in citrus fruits, isoflavones (e.g., genistein) found in soybeans, and flavonols (e.g., quercetin) found in onions and apples. FIG. 1 shows the chemical structures of certain exemplary phytochemicals. Table 1 shows additional exemplary phytochemicals, the plants from which they were isolated, and the observed antimicrobial or anticancer activity. One skilled in the art can readily obtain isolated phytochemicals for use in the present invention.

TABLE 1
Exemplary Phytochemicals

| Common name | Scientific name | Compound | Class | Activity |
|---|---|---|---|---|
| Alfalfa | Medicago sativa | ? | | Gram-positive organisms |
| Allspice | Pimenta dioica | Bugenol | Essential oil | General |
| Aloe | Aloe barbadensis, | Latex | Complex mixture | Corynebacterium, Salmonella, |
| | Aloe vera | | | Streptococcus, S. aureus |
| Apple | Molus sylvestris | Phloretin | Flavenoid derivative | General |
| Ashwagandha | Withania somniferum | Withafarin A | Lactone | Bacteria, fungi |
| Aveloz | Euphorbia thrucalli | ? | | S. aureus |
| Bad tree | Aegle marmelos | Essential oil | Terpenoid | Fungi |
| Balsam pear | Momordica charantia | ? | | General |
| Barberry | Berberis vulgaris | Berberine | Alkaloid | Bacteria, protozoa |
| Basil | Ochmum basilicum | Essential oils | Terpenoids | Salmonella, bacteria |
| Bay | Laurus nobilis | Essential oils | Terpenoids | Bacteria, fungi |
| Betel pepper | Piper betel | Catechols, euenol | Essential oils | General |
| Black pepper | Piper nigrum | Piperdine | Alkaloid | Fungi, Lactobacillus, |
| | | | | Micrococcus, E. coli |
| | | | | E. facecalis |
| Blueberry | Vacctnium spp. | Fructose | Monosaccharide | E. coli |
| Brazilian pepper tree | Schirius terebinthifolius | Terebinthone | Terpenoids | General |
| Buchu | Barosma setulina | Essential oil | Terpenoid | General |
| Burdock | Arctium lappa | | Polymoetylone, tannins, | Bacteria, fungi, viruses |
| | | | terpenoids | |
| Buttercup | Ranunculus bulbosus | Protoanemonia | Lactone | General |
| Caraway | Carum carvt | | Cournarius | Bacteria, fungi, viruses |
| Cascara sagrade | Rhamnus purshiana | Tannins | Polyphenols | Viruses, bacteria, fungi |
| | | | Anthraquinone | |
| Cashew | Anacardium pudsatilla | Salicylic acids | Polyphenols | P. acnes |
| | | | | Bacteria, fungi |
| Castor bean | Ricinus communis | ? | | General |
| Ceylon cinnamon | Cinnamornum verum | Essential oils, | Terpenoids, tannins | General |
| | | others | | |
| Charmomile | Matricaria charmomilla | Anthemic acid | Phenolic acid | M. tuberculosis, S. typhi- |
| | | | | murium, S. aureus, |
| | | | | holminths |
| | | — | Coumarins | Viruses |
| Chapparal | Larnea tridentate | Nordihydrogual- | Lignan | Skin bacteria |
| | | aretic acid | | |
| Chili peppers, | Capricum annuum | Capesicin | Terpenoid | Bacteria |
| paprike | | | | |
| Clove | Syzyglum aromaticum | Eugonol | Terpenoid | General |
| Coca | Erythraxylum coca | Cocaine | Alkanoid | Gram-negative and -positive |
| | | | | cocci |
| Cockle | Agrostemma githago | ? | | General |
| Coltafoot | Tussilao farfara | ? | | General |
| Coriander, cilantro | Corlandrum sativum | ? | | Bacteria, fungi |
| Cranberry | Vaccinlum spp. | Fructose | Monosaccharide | Bacteria |
| | | Othere | | |
| Dandelion | Tararacum officinals | ? | | C. albicans, S. cerevisiae |
| Dill | Anethum graveolens | Essential oil | Terpenoid | Bacteria |
| Echinacea | Echinaceae | ? | | General |
| | angustifolia | | | |
| Eucalyptus | Eucalyptus globules | Tannin | Polyphenol | Bacteria, viruses |
| | | — | Terpenoid | |
| Fava beans | Vicia faba | Pebatin | Thionin | Bacteria |
| Gamboges | Garcinia hanburyl | | Resin | General |
| Garlic | Allium satrum | Allicin, ajoene | Sulfoxide | General |
| | | | Sulfated terpenoids | |
| Ginseng | Panaz notoginseng | | Saponins | E. coli, Sporothrix schenckil, |
| | | | | Staphylococcus, Tricho- |
| | | | | phyton |
| Glory lily | Gloriosa superba | Colchicines | Alkaloid | General |
| Goldenseal | Hydrastis Canadensis | Berberine, | Alkaloids | Bacteria, Giardia duodenals, |
| | | hydrastine | | trypanosomes |
| | | | | Platmodia |
| Gotu kola | Centsila asiasica | Asiatocoside | Terpenoid | M. leprae |
| Grapefruit peel | Citrus parodisa | | Terpenoid | Fungi |
| Green Tea | Carnellia sinesis | Catechin | Flavenoid | General |
| | | | | Shigella |
| | | | | Vibrio |
| | | | | S. mutans |
| | | | | Viruses |
| Harmel, rue | Peganum harmala | ? | | Bacteria, fungi |
| Hemp | Canabis sativa | β-Resorcyclic acid | Organic acid | Bacteria and viruses |
| Henna | Lawsonia inermis | Gallic acid | Phenolic | S. aureus |
| Hops | Humulus bupulus | Lupulone, bunanulona | Phenolic acids | General |
| | | — | (Hemi)terpenoids | |
| Horseradish | Armoraela rustlcana | | Terpenoids | General |
| Hyssop | Hyssopus officinalis | — | Terpenoids | Viruses |
| (Japanese) herb | Rabdosia trichocarpo | Trichorabdal A | Terpene | Helicobacter pylori |
| Lantana | Laniane caunara | ? | | General |
| — | Lawsonia | Lawsone | Quinone | M. tuberculosis |
| Lavender-cotton | Santolina chamas- | ? | | Gram-positive bacteria |
| | cyparissus | | | Candida |
| Legume (West | Milleltia thonningil | Alpinumisoflavone | Flavone | Schiatosome |
| African) | | | | |
| Lemon balm | Melissa officinalis | Tannins | Polyphenols | Viruses |
| Lemon verbena | Aloysta triphylla | Essential oil | Terpenoid | Ascaris |
| | | | ? | E. coli, M. tuberculosis, |
| | | | | S. aureus |
| Licroice | Glycyrrkins globra | Glabrol | Phenolic alcohol | S. aureus, M. tuberculosis |
| Lucky nut, yellow | Thonetis peruvlana | ? | | Plasmodium |
| Mace, nutaneg | Myristica fragrans | ? | | General |
| Marigold | Calendula officinalis | ? | | Bacteria |
| Mosquite | Prosopis julifiora | ? | | General |
| Mountain tobacco | Arnica montana | Helanine | Lactones | General |
| Oak | Quercun rubre | Tannins | Polyphenols | |
| | | Querosrin (available | Flavenoid | |
| | | commercially) | | |
| Olive Oil | Olea europaea | Hexemal | Aldehyde | General |
| Onion | Allium cepa | Allicin | Sulfonide | Bacteria, Candida |
| Orange peel | Citrus sinersis | ? | Terpenoid | Fungi |
| Oregon grape | Makonia awuifolia | Berberine | Alkanoid | Plasmodium |
| | | | | Trypansomea, general |
| Pao d'arco | Tabebuis | Sesquiterpenes | Terpenoids | Fungi |
| Papaya | Cartoa papaya | Latex | Mix of terpenoids, | General |
| | | | organic acids, | |
| | | | alkanoids | |
| Pasque-flower | Anemone pulsatilla | Anemosins | Lactone | Bacteria |
| Peppermint | Menthe pipertis | Menthol | Terpenoid | General |
| Periwinkle | Vince minor | Rossepine | Alkanoid | General |
| Peyoto | Lophophone williamill | Mascaline | Alkanoid | General |
| Pointsettia | Euphorbia pulcherriena | ? | | General |
| Poppy | Polygonian aviculare | Opium | Alkanoids and others | General |
| Potato | Solenum tuberosum | ? | | Bacteria, fungi |
| Prostrate knotweed | Polygonsan aviculare | ? | | General |
| Purple prairie clover | Petalosterrum | Petalostemumol | Flavanoid | Bacteria, fungi |
| Quinine | Cinchoma sp. | Quiniae | Alkanoid | Plasmodium spp. |
| Rasvoltia, chandra | Reurolfia serpentine | Roserpine | Alkanoid | General |
| Rosemary | Rosaursekum oficinalis | Essential oil | Terpenoid | General |
| Salafoin | Pmpbrychia violifolia | Tannins | Polyphenols | Ruminal bacteria |
| Sanafras | Satafrae albidum | ? | | Holminths |
| Savory | Setunaja montana | Carvacrol | Terpenoid | General |
| Senma | Cesula angestfolia | Rhein | Anthraquinone | S. aureus |
| Smooth hydrangea, | Hydrendea arborescens | ? | | General |
| never barks | | | | |
| Snakeplant | Rhea coryanboss | ? | | General |
| St. John' wort | Hyparicum perforatum | Hypericin, others | Anthraquinone | General |
| Sweet flag, calamus | Acorus calamus | ? | | Estedo bacteria |
| Tanay | Tanacetum culgore | Essential oils | Terpenoid | Holminths, bacteria |
| Tartagon | Antemizia dracunculus | Caffaio acids, | Terpenoid | Viruses, holminths |
| | | tannins | | |
| | | | polyphenols | |

One of the richest sources for polyphenols is from the tea leaves of Camellia sinensis. The tea leaves contain approximately 40% polyphenols by dry weight. Phenolic phytochemicals for use in the present invention thus include green tea polyphenols, black tea polyphenols, white tea polyphenols, and combinations thereof. For example, the compositions of the invention can comprise green tea powder, black tea powder, white tea powder, and combinations thereof, optionally together with an isolated compound such as epigallocatechin-3-gallate. Tea polyphenols are also known as tea “catechins.” One skilled in the art can readily obtain isolated tea polyphenols for use in the present invention. Other phenolic phytochemicals useful in the present compositions include ginger phenolics, chlorogenic acid, retinoids, carotenoids, narcotics, theaflavins and garlic extract, and mixtures thereof.

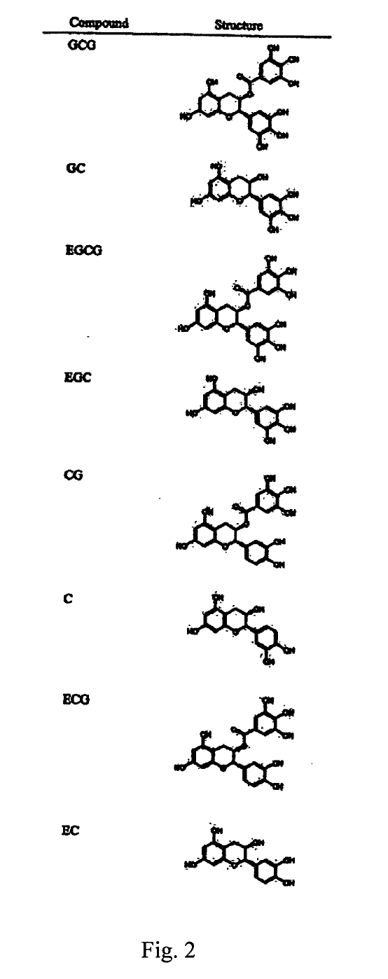

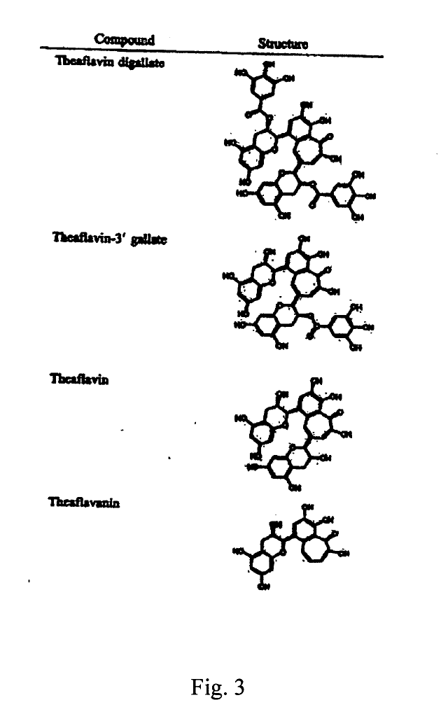

The principal chemical components of green tea polyphenols include epigallocatechin-3-gallate (EGCG), gallocatechin gallate (GCG), gallocatechin (GC), catechin (C), catechin gallate (CG), epicatechin (EC), epicatechin gallate (ECG) and epigallocatechin (EGC), for example as shown in FIG. 2. Such chemical components generally comprise 98% of green tea polyphenols. The principal chemical components of black tea polyphenols are the theaflavins, for example as shown in FIG. 3. Such components generally comprise 98% of black tea polyphenols.

The compositions of the invention can comprise any suitable amount of phytochemical, as can be readily determined by one skilled in the art. For example, if a composition is administered to a subject for the delivery of phytochemicals as a nutritional supplement, the composition can comprise about 0.05 g, about 0.1 g, about 0.25 g, 0.5 g, 1 g or 1.5 g phytochemicals. A composition is administered to a subject for the delivery of phytochemicals as a nutritional supplement can also comprise about 2 wt % to about 50 wt %, for example about 5 wt % to about 20 wt %, of the composition. Greater or lesser amounts are also contemplated. Suitable amounts of phytochemical in compositions administered to a subject for the treatment of cancer or microbial infections are discussed in more detail below.

The compositions of the invention can comprise combinations of phytochemicals. Thus, the present compositions can comprise combinations of pulverized, powdered or dried plant matter containing phytochemicals, optionally in combination with an individual phytochemical, such as an individual polyphenol. As used herein, an “individual phytochemical” is a phytochemical preparation which has been enriched for a given phytochemical, or a phytochemical preparation which is substantially free of other phytochemicals or plant components. For example, a compositions of the invention can comprise pulverized, powdered or dried green tea, pulverized, powdered or dried white tea, and individual EGCG. The green tea and white tea components can be present in the composition in concentrations of about 0.0009 wt % to about 25 wt %. The individual EGCG can be present in the composition in concentrations of about 0.008 wt % to about 0.1 wt % isolated EGCG (about 5% to about 99% pure).

Other components, including other ingredients such as vitamins or minerals (e.g., selenium), probiotics (e.g., yeast), active ingredients such as analgesics, and the like can be included in the compositions of the invention. The compositions of the invention can also comprise chlorophyll, either incorporated into the composition or as a coating.

The compositions of the invention comprising concentrations or combinations different than those found in natural sources can comprise a pharmaceutically acceptable controlled release carrier, or can comprise a pharmaceutically acceptable carrier.

As used herein, a “pharmaceutically acceptable carrier” is any carrier which is suitable for the enteral or parenteral administration to a subject, and includes carriers comprising excipients typically used in the oral care or confectionary industry for formulating oral care compositions or confections.

Suitable pharmaceutically acceptable carriers include consumable drinks; dentifrices (including pastes, gels and liquids for cleaning teeth); mouth washes and oral rinses; dental flosses; and orally consumable films; as are known in the art. Such pharmaceutically acceptable carriers can comprise excipients such as fluoride ion sources, additional anticalculus agents, buffers, other abrasive materials, peroxide sources, alkali metal bicarbonate salts, thickening materials, humectants, water, surfactants, titanium dioxide, flavor system, sweetening agents, xylitol, coloring agents, and mixtures thereof. Techniques for formulating pharmaceutically acceptable carriers according to the invention are within the skill in the art; for example as described in U.S. Pat. Nos. 6,740,311 and 6,689,342, the entire disclosure of which are herein incorporated by reference.

As used herein, a “pharmaceutically acceptable controlled release carrier” means any carrier which is suitable for enteral or parenteral administration to a subject, and which allows controlled release of a phytochemical. For example, the pharmaceutically acceptable controlled release carrier can comprise a gum product, a lollipop or other dosage form comprising a holder, a lozenge, mints (including pressed mints), low boiled candy, hard boiled candy, coated candy, throat drops and the like, or a suppository, as is known in the art. A pharmaceutically acceptable controlled release carrier of the invention can comprise excipients or additives typically used in the oral care or confectionary industry, for example flavorants. Suitable flavorants include peppermint oil, menthol, spearmint oil, vanilla, cinnamon, wintergreen oil, fruit flavorings including (e.g., lemon oil, orange oil, grape flavor, lemon oil, grapefruit oil, apple, apricot essence, and combinations thereof), and combinations of the foregoing. Techniques for formulating a pharmaceutically acceptable controlled release carrier according to the invention are within the skill in the art; see, e.g., U.S. Pat. No. 6,511,679, the entire disclosure of which is herein incorporated by reference.

As used herein, “controlled release” of a phytochemical means that the phytochemical is released from the composition over time upon administration of the composition to the subject. For example, the phytochemical can be released from the composition for at least about one minute, at least about five minutes, at least about ten minutes, at least about 15 minutes, at least about 30 minutes, at least about 45 minutes or at least about 60 minutes following administration. The phytochemical can be released for greater or lesser periods of time following administration. Controlled release of the phytochemical can occur continuously over a given time period, or can occur discontinuously over a given time period. It is understood that a composition of the invention can exhibit both continuous and discontinuous controlled release of a phytochemical at different times after administration to a subject.

Suitable pharmaceutically acceptable controlled release carriers also include various chewable gum (also called “chewing gum”) formulations. Chewing gum formulations permit the controlled release of the phytochemical as the gum product is masticated, or chewed. The action of saliva on the gum can further facilitate release of a phytochemical, as well as its subsequent absorption by the mucous membranes lining the mouth, throat, larynx and esophagus.

The chewing gum of the present invention comprises at least one phytochemical and a gum base. The gum base can include at least one gum base material which can be selected from the many water- and saliva-insoluble gum base materials known in the art. Suitable gum base materials include polymers, such as natural and synthetic elastomers and rubbers, as well as mixtures thereof. Naturally-derived polymers include substances of plant origin like chicle, jelutong, gutta percha and crown gum; and synthetic elastomers such as butadiene-styrene copolymers, isobutylene and isoprene copolymers (e.g., “butyl rubber”), polyethylene, polyisobutylene, polyvinylesters such as polyvinylacetate, and mixtures of any of the foregoing.

The gum base can be selected so as to provide a final chewing gum composition which has a relatively “soft” chew both at the onset of mastication, as well as towards the end of the chewing process (typically about 20 to 30 minutes). Another characteristic of the gum base can be its ability to facilitate controlled release of a phytochemical during the time in which the chewing gum is chewed, in particular during about the first 5 to about 10 minutes after administration.

The chewing gum can comprise material which has hydrophilic characteristics, such as low to medium weight polyvinylacetate (e.g., polyvinylacetate having a number-average molecular weight of about 12,000 to about 45,000). The amount of polyvinylacetate in the gum base can be maximized with no butyl rubber present, and the quantity of non-polyvinylacetate polymers such as butadiene-styrene, butylene-based polymers and copolymers can be minimized. Inclusion of polyvinylacetate can provide a gum base which yields a softer, less brittle and less sticky gum composition, thereby contributing to a more organoleptically pleasing chewing sensation. Polyvinylacetate also tends to be more hydrophilic in nature, and may allow for better release of the saliva-soluble phytochemicals from the gum composition.

A gum base for use in the invention can comprise from about 25 wt % to about 90 wt %, for example 30 wt % to about 75 wt %, about 50 wt % to about 60 wt %, or about 55 wt % of the total chewing gum composition. It is understood that too much gum base may interfere with the release of the phytochemical, and additionally may contribute to tackiness and poor mouth-feel of the final composition.

The gum base can comprise other ingredients such as plasticizers and softeners to help reduce the viscosity of the gum base to a desirable consistency, and to improve the overall texture and bite. Suitable plasticizers and softeners included lecithin; mono- and diglycerides; lanolin; stearic acid; sodium stearate; potassium stearate; glycerol triacetate; glycerol monostearate and glycerin. Without wishing to be bound by any theory, plasticizers and softeners appear to facilitate release of the phytochemical upon mastication. Plasticizers and softeners can comprise from about 0.1 wt % to about 20 wt %, for example about 5 wt % to about 15%, of the gum base.

The gum base can also comprise waxes such as beeswax and microcrystalline wax, and fats/oils such as soybean and cottonseed oils. Such waxes also function as softening agents. Typically, these compounds (either alone or in combination) can comprise from about 0 wt % up to about 25 wt %, for example about 15 wt % to about 20 wt % or less than about 20 wt % of the gum base.

The gum base can also comprise elastomer solvents, including rosin and resin material typically utilized in the confectionery chewing gum industry. Suitable elastomer solvents include methyl, glycerol, and pentaerythritol esters of rosins or modified rosins, such as hydrogenated, dimerized or polymerized rosins or mixtures thereof. Such rosins include pentaerythritol ester of partially hydrogenated wood rosin; pentaerythritol ester of wood rosin; glycerol ester of wood rosin; glycerol ester of partially dimerized rosin; glycerol ester of polymerized rosin; glycerol ester of tall oil rosin; glycerol ester of wood rosin; partially hydrogenated wood rosin and partially hydrogenated methyl ester of rosin, such as polymers of alpha-pinene or beta-pinene, and terpene resins including polyterpene; and mixtures thereof. Elastomer solvents can comprise from about 0.1 wt % to about 75 wt %, for example not more than about 10 wt % of the gum base.

The gum base can also comprise filler material, which can enhance the “chewability” of the chewing gum composition. Suitable filler materials include metallic mineral salts such as calcium carbonate; magnesium silicate (talc); dicalcium phosphate; alumina; aluminum hydroxide; aluminum silicates; and mixtures thereof. Filler material will typically comprise about 0.1 wt % to about 30 wt %, for example about 10 wt % to about 20 wt %, of the gum base.

The gum base can also comprise trace amounts (e.g., less than about 0.1 wt % of the gum base) of standard industry preservatives such as butylated hydroxy toluene (BHT).

The gum base can also comprise at least one sweetener, which can be added to impart improved palatability to the chewing gum composition. It is understood that the sweetener may or may not be perceptibly sweet. Suitable sweeteners include saccharides such as sucrose, glucose (e.g., corn syrup), dextrose, invert sugar, fructose (including high-fructose corn syrup), maltodextrin, and polydextrose; saccharin and its various salts such as the sodium and calcium salts; cyclamic acid and its various salts; dipeptide sweeteners; chlorinated sugar derivatives such as sucralose, dihydrochalcone, glycyrrhin, Stevia rebaudiana (Stevioside); and sugar alcohols such as sorbitol, sorbitol syrup, mannitol, xylitol, hexa-resorcinol and the like; hydrogenated starch hydrolysate, (lycasin); the potassium, calcium and sodium salts of 3,6-dihydro-6-methyl-1-1,2,3-oxathiazin-4-on3-2,2-dioxide; and mixtures of any of the foregoing. The sweetener can comprise about 20 wt % to about 75 wt %, for example about 25 wt % to about 40 wt %, or about 30 wt % to about 35 wt %, of the gum base.

The gum base can also comprise one or more flavoring or coloring agents. These may be selected from any of the industry-available natural- and synthetically-derived food and pharmaceutical flavors or coloring agents. For example, the flavoring agents can impart a cooling or vaporizing sensation to the subject upon mastication of the chewing gum composition. Suitable flavoring agents include oils of peppermint; spearmint; wintergreen; cinnamon; menthol; and menthone; derivatives of such oils; and combinations thereof. Food and pharmaceutical grade coloring agents available throughout the industry can also be utilized. Flavor and coloring agents, either alone or in combination, can comprise from about 0.1 wt % to about 10 wt %, for example about 0.5 wt % to about 5 wt %, or about 2 wt % to about 3 wt %, of the chewing gum composition.

The chewing gum or other controlled release formulation of the invention can also comprise an encapsulated phytochemical. Encapsulation can impart a greater degree of stability to the phytochemical during relatively prolonged periods of commercial storage. Encapsulating a phytochemical can also further enhance the hydrophilicity of less water-soluble versions of the compounds, and can also act to regulate the dissolution of the more highly soluble forms of the compounds. Encapsulation can be accomplished by methods within the skill in the art, for example by employing one or more edible food-grade materials as processing aids. Such edible materials can include oleaginous substances (fats and oils), as well as saccharides, proteins and other non-toxic polymeric material, especially those with emulsifying properties. Without wishing to be bound by any theory, it is believed that oleaginous or other encapsulating material surrounds and enrobes the phytochemical, thereby creating a matrix of several thousand or more individually enrobed particles that can be combined into a chewing gum composition of the invention.

Suitable oleaginous encapsulating materials include various food-grade oils and fats available in the industry, such as stearine; canola, cottonseed and soybean oils; medium chain triglyceride (MCT) oils and mono-, di- and triglyceride-based fatty acid oils. The encapsulating material can comprise about 0.1 wt % to about 40 wt %, for example about 0.1 wt % to about 15 wt %, of the gum base.

In use, the chewing gum composition of the invention provides a controlled release of the phytochemical in response to continued chewing, although the rate of the controlled release of the phytochemical may vary. For example, there can be a continuous controlled release of phytochemical for at least about the first 1 to about 5 minutes, and a continuous or discontinuous controlled release of phytochemical over the next about 5 to about 30 minutes a rate which is somewhat lower than that attained during the initial about 1 to about 5 minutes. About 60%, for example about 80%, about 90%, about 95% or about 100% of phytochemical content can be released from the chewing gum composition within about 20-30 minutes of administration to a subject. It is understood that the release of phytochemical can be substantially independent of the actual chew rate, and that phytochemical release can occur whether or not the chewing gum composition is chewed continuously over a given period of time.

The chewing gum compositions described above can be formulated into any desired shape or size, as is within the skill in the art. For example, the composition can take the shape of sticks or tabs, or any other form which is typically utilized by chewing gum manufacturers. See, e.g., U.S. Pat. Nos. 4,647,450; 5,087,460; 4,792,453; and 5,248508, the entire disclosures of which are herein incorporated by reference.

The chewing gum formulations described above can be prepared using methods known in the confectionery industry for preparing commercial chewing gums; see, e.g., U.S. Pat. Nos. 4,405,647; 5,431,929; 5,736,135; 5,922,347; 5,912,030; 5,866,179; 5,824,291; 5,834,002; 5,846,557; and 5,569,477, the entire disclosures of which are herein incorporated by reference. For example, the gum base can be melted or softened using one or more of the softening agents, plasticizers and/or solvent and filler materials described above. Sweeteners and flavors, whether processed via flash-flow processing or other traditional mixing methods, are then admixed into the gum base. This is accomplished by comminuting the gum base material together with the water-soluble ingredients in a bed or blender within a gaseous medium at room temperature, as described in, e.g., U.S. Pat. No. 4,405,647, supra. This material is continuously pulverized and thereby chopped into much smaller particles. To prevent adherence of the resultant particles to one another, additional filler or bulking material may be added, such as silica gel or calcium carbonate. Granules of any desired size and shape can be obtained by sieving with a standard mesh screen.

The final chewing gum composition is formed by adding the phytochemical to the formed particulates. This is done by admixing the phytochemical, whether in free form or encapsulated as described above, with the pulverized materials so as to substantially disperse the phytochemical among the particulates. The phytochemical thus becomes substantially entrapped in the multitude of spaces between the individual gum particles.

The chewing gum composition of the invention can comprise various centerfill configurations, as are known in the art. In such configurations, the gum base will at least partially surround a centerfill portion comprising one or more phytochemicals. The gum base in such centerfill configurations can also comprise one or more phytochemicals, as described above. The centerfill portion can be a liquid or semi-liquid material, and can comprise one or more sweeteners and/or flavorants as described above. A centerfill configuration may be desirable when an immediate initial release of the phytochemical is desired, such as when treating cancer or microbial infection of the upper gastrointestinal tract.

A chewing gum composition of the invention comprising a centerfill configuration can be prepared using methods known in the confectionery and chewing gum industries. For example, U.S. Pat. No. 3,806,620, the entire disclosure of which is herein incorporated by reference, describes a method for forming centerfill chewing gum. Other methods of forming centerfill chewing gum known in the art may also be utilized, such as are described in U.S. Pat. Nos. 4,250,196; 4,513,012; 4,316,915; 4,292,329; and 4,642,235, the entire disclosures of which are herein incorporated by reference.

Many phytochemicals, for example green tea polyphenols (GTPPs), are antioxidants. The ability of such compounds to scavenge reactive oxygen species such as hydrogen peroxide (H₂O₂) and superoxide radicals is apparently a function of the phenolic groups. Therefore GTPPs, especially epigallocatechin-3-gallate (EGCG), may protect normal cells from chemical or physical damage that may lead to carcinogenesis. The compositions of the invention can therefore be used to deliver phytochemicals to a subject as nutritional or dietary supplements.

Conversely, GTPPs can induce cytotoxicity and apoptosis in many types of tumor cells. For example, EGCG-induced apoptosis has been associated with oxidative stress imposed on tumor cells, especially by H₂O₂. EGCG-induced production of H₂O₂ was recently observed under in vitro conditions in cells and in cell-free systems. Without wishing to be bound by any theory, it is believed that EGCG-induced oxidative stress can trigger an apoptotic pathway that is distinct from chemical or Fas-mediated apoptotic pathways, perhaps through activation of the MAP kinases c-Jun N-terminal kinase and p38. The compositions of the invention can therefore be used to deliver phytochemicals for the treatment or prevention of cancer in a subject. The compositions of the invention can also be used to deliver phytochemicals for the treatment or prevention of microbial infections in a subject.

In the practice of the present methods, an effective amount of one or more phytochemicals is delivered to a subject in need of treatment for cancer or microbial infections. As used herein, an “effective amount” of one or more phytochemicals is an amount sufficient to inhibit proliferation of cancer cells or microbial pathogens in a subject. One skilled in the art can readily determine an effective amount of a phytochemical to be delivered to a given subject, by taking into account factors such as the size and weight of the subject; the extent of the tumor growth, infection or disease penetration; the age, health and sex of the subject; the route of administration; and whether the administration is regional (e.g., local) or systemic.

For example, an effective amount of one or more phytochemicals can comprise from about 1 mg to about 3000 mg compound/kg of body weight, for example between about 10 mg to about 1000 mg or at least about 100 mg compound/kg, of body weight. It is contemplated that greater or lesser amounts of a phytochemical can be administered to a subject. An effective amount of a phytochemical can also be based on the approximate weight of a tumor mass to be treated. The approximate weight of a tumor mass can be determined by calculating the approximate volume of the mass, wherein one cubic centimeter of volume is roughly equivalent to one gram. An effective amount of a phytochemical based on the weight of a tumor mass can be at least about 10 mg/gram of tumor mass, for example between about 1-500 mg/gram or at least about 60 mg/gram, or at least about 100 mg/gram, of tumor mass. It is contemplated that greater or lesser amounts of a phytochemical can be administered to a subject. An effective amount can also be based on the weight percent of phytochemical in the composition. For example, an effective amount can comprise about 2 wt % to about 50 wt %, for example about 5 wt % to about 20 wt %, of the composition. Greater or lesser wt % amounts are also contemplated

One skilled in the art can also readily determine an appropriate dosage regimen for administering the present compositions to a subject. For example, the compositions can be administered to the subject once. Alternatively, the agent can be administered once or twice daily to a subject for a period of from about one to about twenty-eight days, more preferably from about seven to about ten days.

Cancers which can be treated or prevented with the present methods include cancers of at least the following histologic subtypes: sarcoma (cancers of the connective and other tissue of mesodermal origin); melanoma (cancers deriving from pigmented melanocytes); carcinoma (cancers of epithelial origin); adenocarcinoma (cancers of glandular epithelial origin); cancers of neural origin (glioma/glioblastoma and astrocytoma); and hematological neoplasias, such as leukemias and lymphomas (e.g., acute lymphoblastic leukemia and chronic myelocytic leukemia).

Cancers which can be treated or prevented with the present methods also include cancers having their origin in at least the following organs or tissues, regardless of histologic subtype: breast; tissues of the male and female urogenital system (e.g., ureter, bladder, prostate, testis, ovary, cervix, uterus, vagina); lung; tissues of the gastrointestinal system (e.g., stomach, large and small intestine, colon, rectum); exocrine glands such as the pancreas and adrenals; tissues of the mouth and esophagus; brain and spinal cord; kidney (renal); pancreas; hepatobiliary system (e.g., liver, gall bladder); lymphatic system; smooth and striated muscle; bone and bone marrow; skin; and tissues of the eye (e.g., retinoblastomas).

Cancers which can be treated or prevented with the present methods further include cancers or tumors in any prognostic stage of development, for example as measured by the “Overall Stage Groupings” (also called “Roman Numeral”) or the “Tumor, Nodes, and Metastases” (TNM) staging systems. Appropriate prognostic staging systems and stage descriptions for a given cancer are known in the art, for example as described in the National Cancer Institute's “CancerNet” Internet website.

The microbial infections which can be treated or prevented by the present methods include Streptococcal, Staphylococcal, Coliform and Helicobacter infections; tuberculosis; dysentery; cholera; anthrax; bacterial meningitis and oral bacterial infections (e.g., dental caries).

One skilled in the art can evaluate treatment of cancer or microbial infection by the present method by determining whether proliferation of cancer cells or microbial pathogens in the subject has been inhibited. As used herein, to “inhibit the proliferation of cancer cell or microbial pathogen” means to kill the cancer cell or microbial pathogen, or permanently or temporarily arrest the growth of the cancer cell or microbial pathogen. Inhibition of cancer cell or microbial pathogen proliferation can be inferred if the number of cancer cells or microbial pathogens in the subject remains constant or decreases after administration of a the present composition. An inhibition of cancer cell proliferation can also be inferred if the absolute number of cancer cells increases, but the rate of neoplasm or tumor growth decreases.

The number of microbial pathogens in a subject can be determined by standard clinical techniques, such as by bacterial culture. Inhibition of microbial pathogen growth can also be inferred by observing a subject's clinical symptoms of microbial pathogen infections, wherein an improvement in such symptoms indicates an inhibition of microbial pathogen growth.

The number of cancer cells in a subject's body can also be determined by standard clinical techniques, such as by direct measurement or by estimation from the size of primary or metastatic tumor masses. The size of a tumor mass can be ascertained, for example, by direct visual observation or by diagnostic imaging methods such as X-ray, magnetic resonance imaging, ultrasound, and scintigraphy. Such diagnostic imaging methods can be employed with or without contrast agents, as is known in the art. The size of a tumor mass can also be ascertained by physical means, such as palpation of the mass or measurement of the mass with a measuring instrument such as a caliper.

The compositions of the invention can be formulated according to well-known techniques for administration by any enteral or parenteral route, including oral, buccal, sublingual, rectal, parenteral, topical, inhalational, injectable and transdermal, using standard techniques.

The invention will now be illustrated with the following non-limiting example.

An exemplary composition of the invention contains from about 0.0009 wt % to about 25 wt % green tea and/or white tea powder in a gum base. The composition further comprises from about 0.008 wt % to about O. lwt % isolated EGCG in concentration from about 5% to about 99%. For example, an approximately 120 mg total weight chewable composition can contain from about 0.108 mg to about 24 mg of green tea powder and/or white tea powder; about 0.1 mg to about 2 mg of about 5 to about 99% isolated EGCG; and optionally has a chlorophyll coating. Results show that the addition of green tea powder and/or white tea powder past about 25 wt % inhibits chewing and production of the chewable gum composition.

In other chewable gum compositions, green tea polyphenol is present as green tea powder in a range of about 5 wt %, about 20 wt %, or about 12 wt % of the composition. In this example, the green tea powder is present in a range of about 10 gg to about 24 gram within the composition, and contains EGCG in an amount of about 2 wt % to about 50 wt % of the green tea powder. The composition may also optionally contain chlorophyll. The chlorophyll aids in preventing oxidation of the composition and also aids in coloring the composition. As previously indicated the composition may optionally contain white tea extracts instead of, or along with, the green tea powder. The gum base is formed below 120 degrees Fahrenheit. The green tea polyphenol is continuously delivered over at least a 20-minute time period upon administration to a subject. The composition is administered to a subject at least two times a day as a nutritional supplement, or for treating oral cavity disorders including oral cancers, and microbial infections in the mouth and teeth.

All documents referred to herein are incorporated by reference. While the present invention has been described in connection with certain illustrated examples and the various tables and figures, it is to be understood that other similar examples may be used or modifications and additions made to the described examples for performing the same function of the present invention without deviating therefrom. Therefore, the present invention should not be limited to any single example, but rather should be construed in breadth and scope in accordance with the recitation of the appended claims.

I claim:

1. A composition comprising a phytochemical and an pharmaceutically acceptable controlled release carrier.

2. The composition of claim 1, wherein the pharmaceutically acceptable controlled release carrier comprises a chewable gum.

3. The composition of claim 1, wherein the pharmaceutically acceptable controlled release carrier comprises a lozenge, mints, low boiled candy, hard boiled candy, coated candy, throat drops, or a suppository.

4. The composition of claim 1, wherein the pharmaceutically acceptable controlled release carrier comprises a holder.

5. The composition of claim 1, further comprising chlorophyll.

6. The composition of claim 1, wherein the composition releases the phytochemical for at least five minutes upon administration to a subject.

7. The composition of claim 1, wherein the phytochemical is continuously released.

8. The composition of claim 1, wherein the phytochemical is a phenolic phytochemical.

9. The composition of claim 8, wherein the phenolic phytochemical is a green tea polyphenol, black tea polyphenol or white tea polyphenol.

10. The composition of claim 8, wherein the phenolic phytochemical is a combination of green and white tea polyphenols; black and white tea polyphenols; or green, white and black tea polyphenols, optionally in combination with an individual phytochemical.

11. The composition of claim 8, wherein the phenolic phytochemical is selected from the group consisting of ginger phenolics, chlorogenic acid, retinoids, carotenoids, narcotics, theaflavins and garlic extract.

12. The composition of claim 8, wherein the phenolic phytochemical is selected from the group consisting of epigallocatechin-3-gallate (EGCG), gallocatechin gallate (GCG), gallocatechin (GC), catechin (C), catechin gallate (CG), epicatechin (EC), epicatechin gallate (ECG) and epigallocatechin (EGC).

13. The composition of claim 1, comprising about 5 wt % to about 20 wt % of the phytochemical.

14. A chewable gum comprising about 0.0009 wt % to about 25 wt % green tea powder or white tea powder.

15. The chewable gum of claim 14 comprising about 5% to about 95% epigallocatechin-3-gallate (EGCG) that is about 0.008 wt % to about 0.1 wt % of said chewable gum.

16. The chewable gum of claim 14, further comprising chlorophyll.

17. A method of treating cancer or microbial infection in a subject, comprising the step of administering a composition of claim 1 to the subject, wherein the composition of claim 1 delivers an effective amount of phytochemical to the subject.

18. The method of claim 17, wherein the composition administered orally.

19. The method of claim 17, wherein the composition comprises a chewable gum.

20. The method of claim 17, wherein the composition is administered to the subject at least twice in a twenty-four hour period.

21. A method of inhibiting growth of cancer cells or microbial pathogens in a subject, comprising the step of administering a composition of claim 1 to the subject, wherein the composition of claim 1 delivers an effective amount of phytochemical to the subject.

22. The method of claim 21, wherein the composition is administered orally.

23. The method of claim 21, wherein the composition comprises a chewable gum.

24. The method of claim 21, wherein the composition is administered to the subject at least twice in a twenty-four hour period.

25. A method of delivering a phytochemical to a subject as a nutritional supplement, comprising administering a composition of claim 1 to the subject.

26. A composition comprising a pharmaceutically acceptable carrier and a combination of green tea polyphenols, white tea polyphenols, and an individual phytochemical.

27. The composition of claim 26, wherein the individual phytochemical is an individual polyphenol.

28. The composition of claim 27, wherein the individual polyphenol is selected from the group consisting of epigallocatechin-3-gallate (EGCG), gallocatechin gallate (GCG), gallocatechin (GC), catechin (C), catechin gallate (CG), epicatechin (EC), epicatechin gallate (ECG) and epigallocatechin (EGC).

29. The composition of claim 26, wherein the green tea polyphenols comprise green tea powder.

30. The composition of claim 26, wherein the white tea polyphenols comprise white tea powder.

31. The composition of claim 26, further comprising chlorophyll.

32. The composition of claim 26, wherein the pharmaceutically acceptable carrier is selected from the group consisting of consumable drinks; dentifrices; mouth washes and oral rinses; dental flosses; and orally consumable films.

33. A method of treating cancer or microbial infection in a subject, comprising the step of administering a composition of claim 26 to the subject, wherein the composition delivers an effective amount of the phytochemical to the subject.

34. A method of inhibiting growth of cancer cells or microbial pathogens in a subject, comprising the step of administering a composition of claim 26 to the subject, wherein the composition delivers an effective amount of phytochemical to the subject.

35. A method of delivering a phytochemical to a subject as a nutritional supplement, comprising administering a composition of claim 26 to the subject.
